(19) United States
(12) Patent Application Publication
(10) Pub. No.: US 2006/0276764 A1
(43) Pub. Date: Dec. 7, 2006

(54) Male urinary incontinence device including a cinch and related methods

(76) Inventor: Charles H. Warne, Clermont, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(21) Appl. No.: 11/146,361

(22) Filed: Jun. 6, 2005

(51) Int. Cl. A61F 5/44

(52) U.S. Cl. 604/349; 604/385.09

(57) ABSTRACT

A male urinary incontinence device to be worn by a male user may include an incontinence loop cinch surrounding the male user's penis in an uncinched position and being movable to a cinched position to apply pressure to a urethra of the male user's penis and block urine flow during periods of urinary incontinence. Moreover, the device may further include a moisture-absorbing layer supporting the incontinence loop cinch.

### FIELD OF THE INVENTION

The present invention relates to the field of body fluid or waste material collection, and, more particularly, to urinary incontinence devices and related methods.

### BACKGROUND OF THE INVENTION

Urinary incontinence is a condition in which a patient is unable to naturally control urine flow and unintended leakage occurs. This condition may be the result of disease, injury, or other causes, and it may be both inconvenient and embarrassing to those who suffer from it.

Various prior art devices have been developed for collecting and/or restricting urine flow of male users with urinary incontinence. By way of example, U.S. Pat. No. 6,419,665 to Cohen discloses a male undergarment for urinary incontinence which includes undergarment shorts (e.g., boxers) and a pocket protection cage attached thereto. The cage is made of a resilient plastic material and has an opening at the top, and it is connected to the inside of the front panel of the shorts to allow protrusion of the penis of the wearer into the top opening. The pocket protection cage has portions which are positioned at an angle so that the cage protrudes downwardly into one leg portion of the undergarment. A disposable, flexible and water tight pocket made of an absorbent material is placed in the cage and has a top opening for insertion of the penis. An elastic noose is also provided adjacent the top opening of the cage to help retain the user's penis.

Similar undergarment style incontinence devices are disclosed in U.S. Pat. No. 5,727,568 to Kiser and U.S. Pat. No. 5,984,910 to Berke. The Kiser patent discloses a male incontinence treatment device including an undergarment with an attached flow-restricting device (i.e., belts) which is worn in a position such that the urethra is compressed between the device and the pubic bone of the wearer. The Berke patent discloses a similar undergarment device which has an opening or hole through which the penis passes and is kept upright and close to the body by a flap or strap across the opening. The flap may include an occlusion device integrally incorporated therein and aligned with the opening to be positioned between the base of the shaft of the penis and the scrotum to occlude the urethral passage of the penis. The occlusion device is designed to apply sufficient pressure to occlude the urethral passage without cutting off circulation of blood within the penis through the dorsal vein.

U.S. Pat. No. 6,635,038 to Scovel and U.S. Patent Pub. No. 2004/0106909 to Browning disclose urinary incontinence devices which include a belt or waistband to be worn around the user's waist. The Scovel device includes a urinary bag which hangs from the belt, and the Browning device includes a pocket which hangs from the waistband and has a disposable liner for absorbing urine. Another similar urinary incontinence device is disclosed in U.S. Patent Publication No. 2004/0097893 to Elfstrom et al.

Furthermore, U.S. Pat. No. 6,479,726 to Cole discloses a male urinary incontinence pad which includes an absorbent sheath for wrapping around and enclosing the user's penis. The sheath is made from a continuous piece of water impermeable material lined with a layer of absorbent material. Adhesive tape is used for fastening the sheath around the penis. A similar pad is disclosed in U.S. Pat. No. 6,131,575 to Lenker et al. which includes tabs that wrap around the user's penis and have an adhesive thereon for securing the pad in place.

While such prior art devices may have certain advantages, there is a need for other devices which are relatively simple to use and comfortable to wear, yet provide a user with an effective yet adjustable way to control pressure used for restricting urine flow.

### SUMMARY OF THE INVENTION

In view of the foregoing background, it is therefore an object of the present invention to provide a male urinary incontinence device which is comfortable and provides desired pressure control for restricting urine flow.

This and other objects, features, and advantages in accordance with the present invention are provided by a male urinary incontinence device to be worn by a male user. The male urinary incontinence device may include an incontinence loop cinch surrounding the male user's penis in an uncinched position and being movable to a cinched position to apply pressure to a urethra of the male user's penis and block urine flow during periods of urinary incontinence. Moreover, the device may further include a moisture-absorbing layer supporting the incontinence loop cinch. As such, a user may advantageously selectively apply pressure only during times of urinary incontinence, and release the pressure when urinary control is maintained, for example. Moreover, the moisture-absorbing layer absorbs urinary leakage to help protect clothing, etc.

The moisture-absorbing layer may define an elongated hollow sleeve for receiving the user's penis therein, for example. Moreover, the incontinence loop cinch and the moisture-absorbing pad may be movable relative to one another to apply pressure to the urethra at different locations along the male user's penis.

In addition, the male urinary incontinence device may include an undergarment carrying the moisture-absorbing layer. The moisture-absorbing layer may have an inner surface adjacent the user's penis and an outer surface, and the device may further include a moisture-resistant layer on the outer surface of the moisture-absorbing layer.

The incontinence loop cinch may be carried between the inner and outer surfaces. The incontinence loop cinch may also be carried adjacent the outer surface. A clamp may be carried by the incontinence loop cinch for holding the incontinence loop cinch in the cinched position. Further, the moisture-absorbing layer may be a disposable moisture-absorbing layer, for example.

A method aspect of the invention for treating male urinary incontinence may include positioning an incontinence loop cinch supported by a moisture-absorbing layer to surround a male user's penis in an uncinched position. The method may further include moving the incontinence loop cinch to a cinched position to apply pressure to close a urethra of the male user's penis and block urine flow during periods of urinary incontinence.

### BRIEF DESCRIPTION OF THE DRAWINGS

FIG. 1 is a perspective view of a male urinary incontinence device in accordance with the present invention.

FIG. 2 is a cross-sectional diagram of the male urinary incontinence device of FIG. 1 taken along the line 2-2.

FIG. 3 is side view of an alternate embodiment of a male urinary incontinence device including a clamp carried by the incontinence loop cinch in accordance with the present invention.

FIG. 4 is a top plan view of an alternate embodiment of a male urinary incontinence device in accordance with the present invention.

FIG. 5 is a cross-sectional view of the male urinary incontinence device of FIG. 4 taken along the line 5-5.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout, and prime and multiple prime notation are used to indicate similar elements in alternate embodiments.

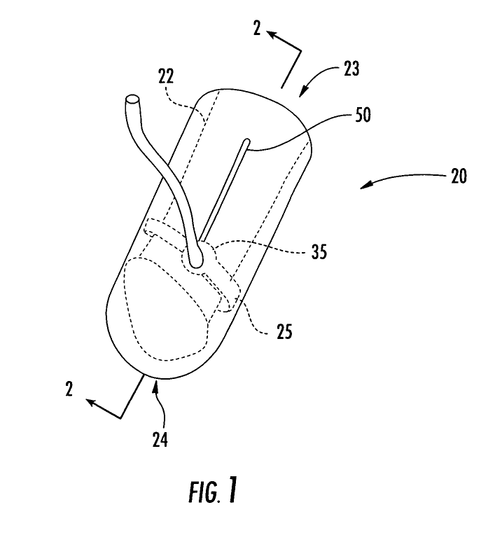

Referring initially to FIGS. 1 and 2, a male urinary incontinence device 20 is for use by a male user with urinary incontinence. The male urinary incontinence device 20 illustratively includes a moisture-absorbing layer 21 which, in the illustrated embodiment defines an elongated hollow sleeve for receiving a user's penis 22 therein. More particularly, the elongated hollow sleeve has an open proximal end 23 through which the user's penis 22 is inserted, and a closed distal end 24.

Furthermore, an incontinence loop cinch 25 is supported or carried by the sleeve and surrounds the user's penis 22 when inserted in the sleeve. The incontinence loop cinch 25 is preferably in an uncinched position when the user is not experiencing urinary incontinence. The incontinence loop cinch 25 is movable to a cinched position to apply pressure to the urethra of the user's penis 22 and block urine flow during periods of urinary incontinence, as will be appreciated by those skilled in the art. As such, the user may advantageously selectively apply pressure only during times of urinary incontinence, and release the pressure when urinary control is maintained, for example. Moreover, the moisture-absorbing layer absorbs urinary leakage to help protect clothing, etc.

It should be noted that since the incontinence loop cinch 25 acts as a tourniquet to restrict urine flow, it may restrict blood flow in the user's penis 22. As such, the user may loosen the incontinence loop cinch 25 to the uncinched position when periods of urinary incontinence are over and urine flow control is reestablished. The uncinched position may be just tight enough to help hold the elongated sleeve in place, but not tight enough to significantly restrict blood flow, for example.

The moisture-absorbing layer 21 may be a disposable moisture-absorbing layer, such as a disposable pad, for example. In other embodiments, the moisture-absorbing layer 21 need not be disposable and may instead be made out of an absorbent fabric (e.g., cotton, etc.) that may be washed and re-used. The moisture-absorbing layer 21 has an inner surface 26 adjacent the user's penis and an outer surface 27. In the illustrated embodiment, perforations 29 may be formed in the moisture-absorbing layer 21 to further aid with absorbency, as will be appreciated by those skilled in the art. Of course, such perforations need not be used in all embodiments.

A moisture-resistant layer 28 may be included on the outer surface. By way of example, the moisture-resistant layer 28 may be made out of plastic or other suitable materials, as will be appreciated by those skilled in the art. In some embodiments, the moisture-resistant layer 28 may be a moisture-repelling chemical or compound, as are also readily known to those skilled in the art.

In the illustrated embodiment, the incontinence loop cinch 25 is positioned between the moisture-absorbing layer 21 and the moisture-resistant layer 28 and held in place with stitching 30. Here, the stitching 30 is spaced apart so that the loop cinch 25 is movable along the length of the male urinary incontinence device 20. As such the user may position the loop cinch in a location providing desired comfort and/or urinary flow restriction. To this end, an opening 50 may be formed in the moisture-resistant layer 28 to allow movement of the external portion of the loop cinch 25 along the length of the device 20, as will be appreciated by those skilled in the art. Of course, other approaches for attaching the incontinence loop cinch 25 to the moisture-absorbing layer 21 may also be used. The drawstring which forms the incontinence loop cinch 25 may be made of various materials including elastic, plastic, fabric, string, etc. In some embodiments, a stationary loop cinch 25 may be used, or multiple loop cinches may be used at different locations along the male urinary incontinence device 20.

In the illustrated embodiment, a loop 35 is at one end of the drawstring, and the other end of the drawstring passes through the loop to form the incontinence loop cinch 25. The loop 35 may be formed in the drawstring, or it may be a ring, eyelet, etc. connected to the drawstring. The loop 35 may have a relatively small diameter or be shaped such that the drawstring fits snugly therein to stay in place in the cinched position. Another approach is to use a noose knot which pulls the drawstring to the cinched position and holds it until loosened by the user.

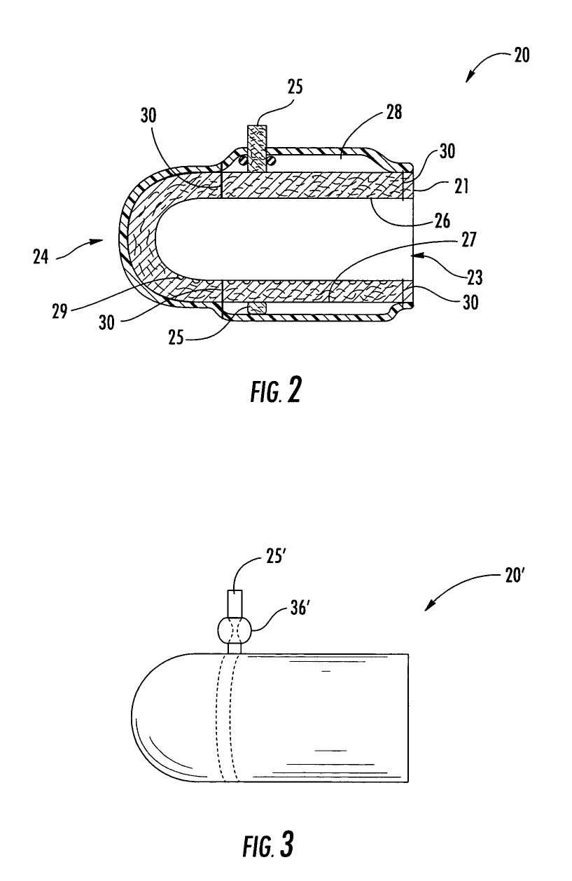

Referring additionally to FIG. 3, a clamp 36′ may also be carried by the incontinence loop cinch 25′ for holding the incontinence loop cinch in the cinched position. In the illustrated example, the clamp 36′ is a bead with a bore therethrough having a smaller diameter than the width of the drawstring. Other suitable clamps and cinching arrangements may also be used, as will be appreciated by those skilled in the art.

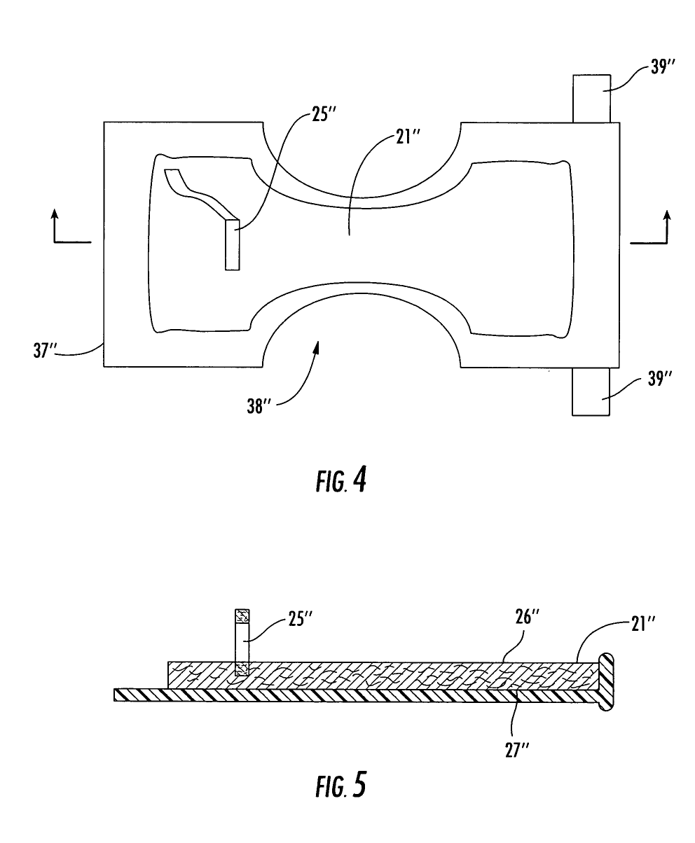

Turning now additionally to FIGS. 4 and 5, in accordance with another embodiment, the male urinary incontinence device 20″ may include an undergarment 37″ carrying the moisture-absorbing layer 21″. In the illustrated example, the undergarment 37″ has an hourglass shape (shown sideways in FIG. 4) with leg holes 38″ and releasable tabs 39″ for securing the undergarment 37″ around the user's waist. Of course, in other embodiments the undergarment 37″ may take other forms, such as brief-style underpants, for example. The undergarment 37″ may also be made of a disposable or re-usable material, as discussed above. In the illustrated example, the incontinence loop cinch 25″ is “sewn” into the moisture-absorbing layer 21″. Other arrangements for attaching the incontinence loop cinch 25″ may also be used, however, as will be appreciated by those skilled in the art. In addition, multiple loop cinches 25″ may be used, or a movable/slidable loop cinch may be used, as discussed above.

A method aspect of the invention for treating male urinary incontinence may include positioning an incontinence loop cinch 25 supported by a moisture-absorbing layer 21 to surround a male user's penis 22 in an uncinched position. The method may further include moving the incontinence loop cinch 25 to a cinched position to apply pressure to close a urethra of the male user's penis 22 and block urine flow during periods of urinary incontinence, as discussed further above.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A male urinary incontinence device to be worn by a male user comprising:
an incontinence loop cinch surrounding the male user's penis in an uncinched position and being movable to a cinched position to apply pressure to a urethra of the male user's penis and block urine flow during periods of urinary incontinence; and
a moisture-absorbing layer supporting said incontinence loop cinch.

2. The male urinary incontinence device of claim 1 wherein said moisture-absorbing layer defines an elongated hollow sleeve for receiving the user's penis therein.

3. The male urinary incontinence device of claim 1 wherein said incontinence loop cinch and said moisture-absorbing pad are movable relative to one another to apply pressure to the urethra at different locations along the male user's penis.

4. The male urinary incontinence device of claim 1 further comprising an undergarment carrying said moisture-absorbing layer.

5. The male urinary incontinence device of claim 1 wherein said moisture-absorbing layer has an inner surface adjacent the user's penis and an outer surface; and further comprising a moisture-resistant layer on the outer surface of said moisture-absorbing layer.

6. The male urinary incontinence device of claim 1 wherein said moisture-absorbing layer has an outer surface and an inner surface, and wherein said incontinence loop cinch is carried between said outer and inner surfaces.

7. The male urinary incontinence device of claim 1 wherein said moisture-absorbing layer has an outer surface and an inner surface, and wherein said incontinence loop cinch is carried adjacent said outer surface.

8. The male urinary incontinence device of claim 1 further comprising a clamp carried by said incontinence loop cinch for holding said incontinence loop cinch in the cinched position.

9. The male urinary incontinence device of claim 1 wherein said moisture-absorbing layer comprises a disposable moisture-absorbing layer.

10. A male urinary incontinence device to be worn by a male user comprising:
an incontinence loop cinch surrounding the male user's penis in an uncinched position and being movable to a cinched position to apply pressure to a urethra of the male user's penis and block urine flow during periods of urinary incontinence;
a moisture-absorbing layer supporting said incontinence loop cinch and defining an elongated hollow sleeve for receiving the user's penis therein, said moisture-absorbing layer having an inner surface adjacent the user's penis and an outer surface; and
a moisture-resistant layer on the outer surface of said moisture-absorbing layer.

11. The male urinary incontinence device of claim 10 wherein said incontinence loop cinch is adjacent the outer surface of the moisture-absorbing layer.

12. The male urinary incontinence device of claim 10 wherein said incontinence loop cinch and said moisture-absorbing pad are movable relative to one another to apply pressure to the urethra at different locations along the male user's penis.

13. A male urinary incontinence device to be worn by a male user comprising:
an incontinence loop cinch surrounding the male user's penis in an uncinched position and being movable to a cinched position to apply pressure to a urethra of the male user's penis and block urine flow during periods of urinary incontinence;
a moisture-absorbing layer supporting said incontinence loop cinch; and
an undergarment carrying said moisture-absorbing layer.

14. The male urinary incontinence device of claim 13 wherein said moisture-absorbing layer has an outer surface and an inner surface, and wherein said incontinence loop cinch is adjacent the outer surface of the moisture-absorbing layer.

15. The male urinary incontinence device of claim 13 wherein said moisture-absorbing layer has an outer surface and an inner surface, and wherein said incontinence loop cinch is carried between said inner and outer surfaces.

16. A method for treating male urinary incontinence comprising:
positioning an incontinence loop cinch supported by a moisture-absorbing layer to surround a male user's penis in an uncinched position; and
moving the incontinence loop cinch to a cinched position to apply pressure to a urethra of the male user's penis and block urine flow during periods of urinary incontinence.

17. The method of claim 16 wherein the moisture-absorbing layer defines an elongated hollow sleeve for receiving the user's penis therein.

18. The method of claim 16 further comprising attaching the moisture-absorbing layer to an undergarment, and putting the undergarment on the male user prior to positioning.

19. The method of claim 16 wherein positioning comprises positioning an inner surface of the moisture-absorbing layer adjacent the user's penis, and wherein the moisture-absorbing layer has an outer surface opposite the inner surface with a moisture-resistant layer thereon.
